(19) United States
(12) Patent Application Publication
(10) Pub. No.: US 2013/0183696 A1
(43) Pub. Date: Jul. 18, 2013

(54) METHODS OF USE AND KIT FOR MEASUREMENT OF LIPOPOLYSACCHARIDE WITH A TIME RESOLVED FLUORESCENCE BASED ASSAY

(71) Applicant: Constance Neely Wilson, Raleigh, NC (US)

(72) Inventor: Constance Neely Wilson, Raleigh, NC (US)

(21) Appl. No.: 13/823,434

(22) PCT Filed: Sep. 15, 2011
(86) PCT No.: PCT/US11/51779

§ 371 Date: Mar. 14, 2013

Related U.S. Application Data
(60) Provisional application No. 61/382,990, filed on Sep. 15, 2010.

(51) Int. Cl. G01N 33/92

(52) U.S. Cl. CPC G01N 33/92
USPC 435/7.92; 436/501; 435/7.1

(57) ABSTRACT

The present invention relates to the methods of use for measurement of lipopolysaccharide (LPS) and methods of use for diagnosis of sepsis and LPS-related conditions. Specifically the present invention relates to a time resolved fluorescence (TRF) based assay for the measurement of LPS and methods of use for the measurement of LPS to diagnose sepsis, Gram-negative bacterial infections, and LPS-related conditions.

This application claims priority of U.S. provisional application No. 61/382,990 filed on Sep. 15, 2010 and is included herein in its entirety by reference.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to the methods of use for measurement of lipopolysaccharide (LPS) and methods of use for diagnosis of sepsis and LPS-related conditions. Specifically, the present invention relates to time resolved fluorescence (TRF) based assay for the measurement of LPS and methods of use for the measurement of LPS to diagnose sepsis and LPS-related conditions.

2. Description of Related Art

Lipopolysaccharide (LPS) is a major component of the outer wall of Gram-negative bacteria and can induce immune responses, including the release of pro-inflammatory cytokines. In sufficiently high concentrations, LPS can induce the release of mediators that produce sepsis, septic shock, and organ damage. Thus, LPS may serve as an important biomarker of sepsis. Moreover, LPS can act as a pyrogen, that is, a fever-inducing substance. Furthermore, LPS released from Gram-negative bacterial infections in organs, such as the eye, urinary bladder, ear, or LPS circulating in the plasma released from Gram-negative bacteria in the bowel, may cause LPS-related conditions, including, cystitis, otitis media, or Alzheimer's disease.

Currently there are only two FDA approved tests to measure LPS which is often referred to as endotoxin. The Limulus amebocyte lysate (LAL) assay is FDA approved for the detection of LPS/endotoxin in human and animal parenteral drugs, biological products, and medical devices for FDA clearance for human use. However, there is a high inherent error rate associated with the LAL test to measure LPS in industrial solutions, including FDA approved products and blood products for human use. Moreover, the LAL endotoxin test is not suitable to measure LPS in biological products, such as antibodies, necessitating the use of the less reliable rabbit pyrogen test for clearance of these products for human use. Furthermore, this test is not FDA approved as a clinical diagnostic to detect or measure LPS/endotoxin in the blood of patients at risk of, or with, sepsis, because a number of interfering substances are present in blood that enhance or inhibit the LAL LPS/endotoxin test causing false positive and false negative test results.

Spectral Diagnostics (Toronto, Canada) has received FDA approval to market its LPS/endotoxin assay, Endotoxin Activity Assay (EAA™). This endotoxin assay is a chemiluminescent test that measures oxygen radical release from neutrophils as an indicator of the level of LPS/endotoxin in blood. This test may lack specificity and is also limited to use in whole blood.

Sepsis is one of the largest unmet medical needs. Sepsis is a medical syndrome characterized by an overwhelming systemic response to infection that can rapidly lead to shock, organ failure, and death. In the U.S., sepsis is the 10th leading cause of death overall and accounts annually for over 750,000 cases, 215,000 deaths, and $17 billion in health care expenditures (Angus et al., Crit Care Med 29:1303-1310; 2001). Moreover, the incidence of sepsis may be rising due to the increasing age of the population, growing numbers of immunocompromised patients, use of life-sustaining technologies, and increased resistance of bacteria to antimicrobial agents. Furthermore, despite advances in critical care medicine, improved antibiotics, and the approval of Eli Lilly and Company's anti-sepsis therapeutic Xigris®, the mortality rate from sepsis is essentially unchanged in over 50 years. Today, therapy for sepsis includes antibiotics, surgical drainage of the site of suspected infection, inotropes and vasopressors to support the heart and blood pressure, and supportive care in an intensive care unit (ICU), including mechanical ventilation. Despite the availability of newer antimicrobial agents and improved supportive care, the mortality rate for severe sepsis and septic shock of 30-60% remains high and the longterm outcome is poor for patients that survive sepsis. These discouraging statistics suggest a need for additional therapies (adjunctive therapies) to this conventional approach to sepsis, therapies that interrupt the complex cascade of events of sepsis leading to shock, multisystem organ dysfunction and failure, and death.

The first FDA-approved adjunctive treatment for sepsis was approved in November 2001. This drug, Xigris (drotrecogin alfa [activated], recombinant human activated protein C), developed and commercialized by Eli Lilly, targets a terminal event in sepsis, disseminated intravascular coagulation (DIC), an abnormality in coagulation leading to severe, diffuse bleeding. In its definitive large clinical trials, Xigris reduced absolute mortality by only 6% compared to placebo (Bernard et al., New Eng J Med 344:699-709, 2001). Moreover, Xigris is expensive and has a serious adverse side effect of bleeding. Because of the unmet medical need for a safe and effective anti-sepsis therapeutic, a number of other pharmaceutical companies have tried and failed to develop adjunctive anti-sepsis therapies, including anti-endotoxin, anti-cytokine, and other anti-sepsis therapies. These attempts have failed to demonstrate a beneficial effect in outcome for a number of reasons. A primary reason for these failures was the lack of a sensitive and specific biomarker/diagnostic to properly identify/stratify patients for the therapies in these clinical trials. Currently there are no reliable, sensitive, and specific biomarkers to identify patients with sepsis, or at risk of sepsis, or safe and effective anti-sepsis therapeutics to treat sepsis.

Over 170 different biomarkers for sepsis, including procalcitonin and C-reactive protein, have been evaluated in clinical trials; however, these biomarkers and the diagnostic tests for them, lack the sensitivity or specificity for routine clinical use by physicians to diagnose sepsis, to identify/stratify patients for a specific anti-sepsis therapy, or for use as a prognostic indicator for organ injury and survival, as well as response to therapy. (Pierrakos and Vincent, Critical Care 14:R15-R33, 2010). To date, there are no reliable clinical diagnostics or biomarkers to identify patients with sepsis, leading to the inclusion of patients with suspected sepsis who present with nonspecific signs and symptoms of a systemic inflammatory response syndrome (SIRS). Not all patients with SIRS have an infection. Currently, a positive bacterial blood culture documenting bacteremia is generally used to diagnose sepsis; however, this test is unreliable, giving a positive result in less than 30% of cases of suspected sepsis.

The EAA is now FDA approved as a clinical diagnostic for detection and measurement of LPS/endotoxin in patients with suspected sepsis. This endotoxin assay is a chemiluminescent test that measures oxygen radical release from neutrophils via complement opsonized LPS-IgM immune complexes. A luminol reaction in the presence of these immune complexes emits light energy. The relative light units (RLU) measured by a luminometer are a measure of LPS in the blood sample and are expressed as a percentage of the total possible activity (0-100%) EA value. In humans, with the use of this LPS assay, EA values less than 0.40 supports the absence of a Gram-negative infection and higher EA values (>0.59) are associated with increased risk of dying while in the ICU. It is believed that this EAA LPS test lacks specificity; it is also limited to use in whole blood and must be performed on site shortly after the blood is drawn, albeit the results are rapid. Roche Diagnostic's LightCycler® SeptiFast test, a polymerase chain reaction (PCR)-based test to detect bacterial and fungal DNA for pathogens in the blood of patients with suspected sepsis is not available in the U.S. Because of its high sensitivity, the false positive rate is high with this test. Finally, based on genomic analysis of samples from patients with sepsis, SIRS-Lab is developing molecular biomarkers for sepsis on a chip, including VYOO®, to measure bacterial and fungal DNA in the blood of patients with sepsis. VYOO is not FDA approved for clinical use in the U.S. Finally, the LAL endotoxin test is not FDA approved for clinical use in the U.S. Lipopolysaccharide (LPS) levels are elevated in the plasma of patients with sepsis (Parsons et al., Am Rev Respir Dis 140:294-301, 1989; Brandtzaeg et al., J Infect Dis 159:195-204, 1989; Danner et al., Chest 99:169-175, 1991). Moreover, LPS binds to and activates A1 adenosine receptors (Wilson and Batra, J Endotoxin Res 8:263-27; 2002). Based on the discovery that LPS/endotoxin binds to A1 adenosine receptors, patents for an assay to measure endotoxin were issued in several countries [U.S. Pat. Nos. 5,773,306, 6,908,742; WO 97/044665; Canada Pat No., 2,253,236; EP 97926695.4 (Austria, France, Germany, Italy, Spain, Switzerland and Lichtenstein, and United Kingdom); Japan Pat. No. 3,197,280]. The current known assay is based on displacement or competition for binding of LPS with a tagged high affinity A1 adenosine receptor ligand, such as BW A844U-biotin, to the A1 adenosine receptor. Based on laboratory and clinical evaluations of this A1 adenosine receptor ligand binding assay to measure LPS in plasma, including the plasma of patients with suspected sepsis, taken together with previous reports, depending on the cut-off value for the LPS measurement, LPS is a sensitive and specific biomarker for patients with suspected sepsis and acute lung injury. However, the high background noise for tags, such as biotin or a fluorescent tag, such as Cy3B, tagged to the competing A1 adenosine receptor ligand, BW A844U, for LPS in this spectrophotometric (or fluorescence) based A1 adenosine receptor ligand binding assay prevented its commercialization. The high background noise, that is, the low signal to noise ratio, resulted in an assay with low reliability and low reproducibility. For clinical use with high commercial value, a sepsis biomarker/diagnostic must meet high sensitivity, specificity, and reliability criteria. Moreover, the assay that measures this biomarker must be user friendly and the results must be provided in a timely manner to physicians caring for patients with suspected sepsis. An assay with high background noise is not reliable, is not reproducible, and cannot be used to measure LPS or be commercially developed. The introduction of time resolved fluorescence (TRF) technologies, including fluorophores with optimal photophysical properties, such as high energy transfer efficiency, and that can be conjugated to biomolecules, as well as improvements in instrumentation and gating techniques over conventional methods of fluorescent measurement, have provided the basis for a new generation of fluorescence-based assays, such as TRF based assays. Time resolved fluorescence (heterogeneous and homogeneous) based assays, including but not limited to TRF, homogeneous TRF (HTRF), time resolved fluorescence resonance energy transfer (TR-FRET), Dissociation-Enhanced Lanthanide Fluorescent Immunoassay (DELFIA®), Time Resolved Amplified Cryptate Emission (TRACE), and Lanthanide Chelate Excite (LANCE®) assays, are sensitive, specific, reliable, robust, user-friendly, have high signal to noise ratios, and can be developed in a number of different formats, including microtiter plate based formats, miniaturized formats, and high throughput assay formats. Moreover, in addition to performing these assays in solution, such as in wells in microtiter plates, these assays can be formatted with the use of a solid phase format, where for example, membranes expressing a G protein coupled receptor (GPCR), such as an A1 adenosine receptor, are coated to a solid phase. Solid phases may include microtiter plates, beads, cards, dipsticks, chips, nanoparticles, and the like.

Accordingly, there is a need for a sensitive, highly accurate, reliable, specific, reproducible, non-radioactive assay with a high signal to noise ratio and low inherent error rate,that can be used to measure LPS in industrial, non-biological solutions, blood products, and biological fluids. Moreover, there is a need for a sensitive and reliable assay with a high signal to noise ratio that can be formatted for clinical use to measure LPS as a biomarker in a patient's plasma or other clinical sample with high specificity for sepsis sufficient to diagnose sepsis or identify patients at risk of sepsis, and to stratify patients with suspected sepsis for an anti-sepsis treatment. Furthermore, there is a need for a sensitive and reliable assay that can be used to measure LPS in clinical samples from patients with LPS-related conditions.

To date, even though there is a great need for such a sensitive and specific sepsis biomarker and assay, there is a large number of sepsis biomarkers known, and there is a plethora of quantitative and qualitative assays known for the last decade or more, none is known or available which can reliably identify patients at risk of sepsis, that have sepsis, or other LPS-related conditions, and can stratify patients for goal-oriented or directed therapy, including treatment with an anti-sepsis therapeutic, such as an anti-LPS therapeutic that blocks the effects of LPS.

### BRIEF SUMMARY OF THE INVENTION

It has now been discovered that LPS activation of the A1 adenosine receptor induces a clear signal in a TRF based assay that is sufficiently sensitive, accurate, and reliable to measure LPS in samples from both biological and non-biological solutions, including water, buffers, other industrial solutions, blood products, and samples from plasma, other body fluids, or clinical sites of suspected infection, from patients at risk of or with sepsis. Moreover, LPS measured with a TRF assay will serve as a sensitive and reliable biomarker and quantitative determinant in patients at risk of sepsis or with sepsis for stratifying patients for specific treatments, including an anti-sepsis therapeutic. Furthermore, LPS measured with a TRF assay will serve as a sensitive and specific biomarker to stratify patients for an anti-LPS therapeutic to treat LPS-related conditions. One particular embodiment includes use of a guanosine triphosphate (GTP) lanthanide chelate and protein source for A1 adenosine receptors in a TRF based binding assay for LPS.

Accordingly, in one embodiment of the present invention, there is a method for measuring quantitative LPS levels in a sample using an A1 adenosine receptor TRF assay comprising:
a) selecting a source for the A1 adenosine receptor protein;
b) selecting at least one TRF fluorophore that is bound to a moiety selected from the group comprising:
   i. a tag;
   ii. a metabolic label;
   iii. a protein label;
   iv. an antibody to a tag, metabolic label, or protein label;
   v. an antibody to an A1 adenosine receptor protein or peptide;
   vi. an A1 adenosine receptor protein or peptide;
   vii. a protein or a peptide in an A1 adenosine receptor signaling pathway;
   viii. an antibody to a protein or a peptide in an A1 adenosine receptor signaling pathway;
   ix. an A1 adenosine receptor ligand;
   x. a signaling molecule;
   xi. a molecule in an A1 adenosine receptor signaling pathway;
   xii. an antibody to a molecule in an A1 adenosine receptor signaling pathway; and
   xiii. a molecule that can be measured in an A1 adenosine receptor signaling pathway; and
c) running the TRF assay utilizing the source for the A1 adenosine receptor protein; the at least one TRF fluorophore that is bound; and the sample in a manner that measurement of fluorescence correlates with the quantitative level of LPS.

In yet another embodiment, there is a method for measuring quantitative LPS levels in a sample using an A1 adenosine receptor TRF assay comprising:
a) selecting a source for the A1 adenosine receptor protein;
b) selecting a first and second TRF fluorophore that is each bound to a different moiety selected from the group comprising:
   i. a tag;
   ii. a metabolic label;
   iii. a protein label;
   iv. an antibody to a tag, metabolic label, or protein label;
   v. an antibody to an A1 adenosine receptor protein or peptide;
   vi. an A1 adenosine receptor protein or peptide;
   vii. a protein or a peptide in an A1 adenosine receptor signaling pathway;
   viii. an antibody to a protein or a peptide in an A1 adenosine receptor signaling pathway;
   ix. an A1 adenosine receptor ligand;
   x. a signaling molecule;
   xi. a molecule in A1 adenosine receptor signaling pathway;
   xii. an antibody to a molecule in an A1 adenosine receptor signaling pathway; and
   xiii. a molecule that can be measured in an A1 adenosine receptor signaling pathway;
   selected such there is energy transfer between the two fluorophores following excitation in a TRF assay which can be measured; and
c) running the TRF assay utilizing the source for the A1 adenosine receptor protein; the first and second TRF fluorophores that are bound; and the sample in a manner that measurement of fluorescence correlates with the quantitative level of LPS.

In another embodiment, there is a method of diagnosing a patient for the presence of sepsis, a Gram-negative bacterial infection, or an LPS-related condition comprising measuring LPS in a biological sample from the patient comprising:
a) selecting a source for an A1 adenosine receptor protein;
b) selecting a quantitative TRF assay that utilizes at least one TRF fluorophore wherein the assay is quantitative for LPS;
c) running the assay with the sample;
d) measuring the amount of fluorescence for the sample in the assay;
e) determining the amount of LPS in the sample from the amount of fluorescence by comparing the fluorescence measurement for the sample to a standard curve for LPS generated by the assay of steps a) through d) with the use of samples spiked with known amounts of LPS; and
f) diagnosing the patient's condition from the LPS amount in the sample.

In yet another embodiment, there is a kit for determination of LPS amount in a sample comprising:
a) a selected TRF assay utilizing at least one fluorophore;
b) a source for the A1 adenosine receptor protein; and
c) an LPS standard.

### BRIEF DESCRIPTION OF THE DRAWINGS

FIG. 1 shows data which represent a standard curve for LPS measured with an A1 adenosine receptor TRF europium (Eu)-GTP binding assay.

### DETAILED DESCRIPTION OF THE INVENTION

While this invention is susceptible to embodiment in many different forms, there is shown in the drawing and will herein be described in detail specific embodiments, with the understanding that the present disclosure of such embodiments is to be considered as an example of the principles and not intended to limit the invention to the specific embodiments shown and described. This detailed description defines the meaning of the terms used herein and specifically describes embodiments in order for those skilled in the art to practice the invention.

### Definitions

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

The term “about” means±10 percent.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawing featured in the figure is for the purpose of illustrating certain convenient embodiments of the present invention, and are not to be considered as limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein “lipopolysaccharide” (LPS) (also known as endotoxin) refers to a glycolipid that is a major component of the outer wall of Gram-negative bacteria. It is normally found in the blood of animals in low concentrations. It is known as an indicator of disease states when the level is elevated in blood and is present in other body fluids, even in low concentrations. It can induce immune responses, including the release of pro-inflammatory cytokines. In sufficiently high concentrations, LPS can induce the release of mediators that produce sepsis, septic shock, and organ damage and failure. Moreover, LPS can act as a pyrogen, that is, a fever-inducing substance. Furthermore, LPS released from Gram-negative bacterial infections in organs, such as the eye, urinary bladder, ear, or LPS circulating in the plasma released from Gram-negative bacteria in the bowel may cause LPS-related conditions. Accordingly, LPS is known as an indicator of the presence or activity of pyrogens, Gram-negative bacterial infection, sepsis, and LPS-related conditions.

The term “protein” as used herein is inclusive of any molecule that contains amino acids, including RNA, RNAi, siRNA, shRNA, miRNA, RNA polymerase, DNA, dsDNA, DNA vectors, DNA fragments, DNA promoters, single nucleotide polymorphisms (SNPs), chromatin, antisenses, oligonucleotides, epitopes, proteins, peptides, polypeptides, enzymes, and the like. The protein may occur in nature, be recombinant in nature, or be genetically engineered. The protein may be solubilized or purified. The protein may be conjugated to a sugar or lipid. It may be linked to another protein to form a protein complex or fusion protein. The protein may be tagged. The protein may be a dimer or a subunit of a protein, such as a subunit of a G protein. Furthermore proteins may be linked to nanoparticles. Sources of protein include but are not limited to mammals, fish, reptiles, plants, insects, bacteria, yeast, and fungi. Sources of protein include tissues and cells or membranes prepared from tissue or cells from plants, yeast, insects, or mammals, such as Chinese Hamster Ovary (CHO) cells or Sf9 insect cells, expressing the protein of choice, such as the A1 adenosine receptor protein.

The term “signaling molecule” as used herein includes but is not limited to any substance with or without tags, including proteins, peptides, oligonucleotides, epitopes, enzymes, kinases, DNA, RNA, anti-sense molecules, phosphatases, thromboxane, interleukins, cytokines, cyclic adenosine monophosphate (cAMP), GTP, nuclear factor kappa beta (NF-κβ), subunits of NF-κβ, inositol triphosphate (IP3), protein kinase C (PKC), diacylglycerol (DAG), heat shock protein, matrix metaloproteinanses, growth factors, and other molecules that can be measured following activation of an A1 adenosine receptor.

The term “sepsis” as used herein is inclusive of sepsis, septicemia (bacteremia/endotoxemia), severe sepsis, septic shock, and related conditions, as well as the clinical symptoms and complications associated with each of these conditions.

The term “LPS-related conditions” include conditions where the level of LPS in a sample correlates with the presence of the condition. For example, 1) in amniotic fluid the level of LPS correlates with the incidence of premature rupture of membranes in pregnancy (Hazan et al., Acta Ostet Gynecol Scand 74:275-280, 1995; 2) in the blood of postoperative patients the LPS level correlates with the incidence of respiratory and renal complications (Berger et al., E Surg Res 28:130-139, 1996); 3) in asthmatics the LPS level in house dust correlates with the severity of asthma (Michel et al., Am J Resp Crit Care Med 154:1641-1646, 1996); 4) in the stool of neonates with necrotizing enterocolitis the LPS level correlates with the severity of bowel mucosal disease (Duffy et al., Digestive Dis and Sci 42:359-365, 1997); 5) in the urine the LPS level correlates with the presence of Gram-negative infection (Hurley and Tosolini, J Microbiol Methods 16: 91-99, 1992); 6) in the culture media for in vitro fertilizations the LPS level correlates with the survival of the embryo (Nagatta and Shirakawa, Fertility and Sterility 65:614-619, 1996); and, 7) in the peritoneal fluid of continuous ambulatory peritoneal dialysis (CAPD) patients the LPS level has 100% sensitivity and specificity for the diagnosis of Gram-negative peritonitis (Ishizaki et al., Advances in Peritoneal Dialysis 12:199-202, 1996). LPS levels in plasma or other patient samples may be associated with other conditions such as neurodegenerative diseases, such as Alzheimer's disease or Parkinson's disease, or diseases resulting in fibrosis and sclerosis, such as inflammation and autoimmune diseases (Jaeger et al., Brain Behav Immun 23:507-517, 2009; Villarán et al., J Neurochem 114:1687-1700, 2010). For further examples and reference to LPS-related conditions, see U.S. Pat. No. 6,117,445, incorporated herein by reference.

Since it takes 24-48 hours to obtain bacterial culture reports for samples from blood or suspected sites of infection in the body, a timely assay that is both sensitive and specific for the detection and measurement of LPS is invaluable in a number of clinical conditions and settings. The measurement of LPS in blood, body fluids, cavities, and tissues, will allow physicians to diagnose the presence of a Gram-negative bacterial infection and prescribe more specific therapy toward Gram-negative bacterial infections earlier, avoiding the use of broad spectrum antibiotics. Moreover, LPS levels in the ear may suggest the presence of a Gram-negative bacterial infection and otitis media, when bacterial cultures of the ear are negative. Furthermore, LPS levels in the cerebral spinal fluid (CSF) may indicate the presence of a Gram-negative bacterial infection and meningitis when CSF bacterial cultures are negative. Also, if LPS levels were detected in peritoneal fluid following major abdominal surgery, this may suggest the presence of an intra-abdominal infection requiring surgical drainage. Finally, it has been reported that certain antibiotics or combinations of antibiotics induce the release of LPS from Gram-negative bacteria (Hurley, Drug Safety 12:183-195, 1995). A sensitive and specific assay for the detection and measurement of LPS would allow investigations into this important area of medicine and the practice of prescribing antibiotics, and possibly influence the development of antibiotic therapies.

Clinical fields of use for a TRF assay to detect and measure LPS include, but are not limited to, a clinical diagnostic for sepsis, Gram-negative bacterial infections, LPS-related conditions, cardiopulmonary bypass surgery, preoperative testing to determine the level of LPS in a sample, such as blood, before surgery, screening blood products, point of care test to diagnose urinary tract infections and asymptomatic bacteriuria of pregnancy, screening dialysis fluids (hepatic and peritoneal), in vitro fertilizations where low (pg/ml) LPS levels correlate with death of the embryo (Nagatta and Shirakawa supra); and organ transplant baths where LPS levels may correlate with failure of the organ transplant.

Nonclinical fields of use for a TRF assay to detect and measure LPS include, but are not limited to, research use only (RUO) and industrial uses. Industrial uses include: screening drugs, medical devices, and biologics for LPS levels as required by the FDA for human use, testing water in cooling systems for Legionella, testing water in humidifiers, including those in line with ventilators, testing water for semiconductor fabrication facilities, testing drinking water, monitoring contact lens solutions, and testing cosmetics for LPS contamination.

Accordingly, the methods and kit of the present invention can be used to determine levels of LPS and diagnose these conditions in an animal. By “animal” it is meant to include but not limited to, mammals, fish, amphibians, reptiles, birds, marsupials and in one embodiment, humans. Thus, by measuring the presence and levels of LPS in a sample from an animal, the conditions above can be rapidly and accurately diagnosed.

As used herein the term “sample” refers to, but is not limited to, biological samples derived from an animal including whole blood, plasma, serum, CSF, urine, saliva, ear fluid, uterine fluid, eye fluid, pleural fluid, peritoneal fluid, bronchoalveolar lavage fluid, pericardial fluid, synovial fluid, sinus fluid, and fluid from cysts, embryo culture media, as well as non-biological samples, such as organ baths, pharmaceutical solutions and products, blood products, medical devices, dialysis fluids, and industrial solutions and products. The sample of the present invention must be in a state that allows testing in a TRF assay so that generally the sample will be in a liquid state such as solution or suspension.

“Time resolved fluorescence assays” involves the use of one or more long-lived fluorophores combined with time-resolved detection (a delay between excitation and emission detection) which allows for detection without major fluorescence interferences. These assays are currently either heterogeneous or homogeneous in nature. The assays of the present invention involve the use of the A1 adenosine receptor. Setting up a TRF assay with the A1 adenosine receptor is within the skill in the art. All GPCRs require different conditions for optimal binding, such as for GTP binding. Therefore, one skilled in the art would optimize the buffer conditions for the specific GPCR, such as the A1 adenosine receptor, as utilized in the present invention. These buffer conditions may differ depending on the protein source for the A1 adenosine receptor, such as a recombinant A1 adenosine receptor stably expressed in cells or membranes from cells,tissues, plants, or bacteria, or solubilized or purified from membranes from cells, tissues, plants, or bacteria, as is known in the art.

TRF assays all have at least one fluorophore bound to a moiety, such as a protein or ligand. Fluorophores useful in the present invention are well known and can easily be selected to be compatible. Where the fluorophore is selected it can be bound, for example, by chelation to a tag, metabolic label, an antibody to the A1 adenosine receptor protein or peptide for the A1 adenosine receptor protein, the A1 adenosine receptor protein or peptide, or the like consistent with the type of assay. In one embodiment the fluorophore is chelated to GTP. One well known TRF binding assay is the GTP binding assay which can be utilized in both heterogeneous and homogeneous TRF assays. Utilizing a GTP-lanthanide moiety typically the fluorescence can be measured at 620 nm. One embodiment of the moiety is GTP chelated to europium (Eu-GTP). The GTP binding assay in general is well known and application to the present invention using the A1 adenosine receptor and LPS is within the skill in the art in view of this disclosure. As used herein the moiety selected to be bound to the fluorophore is selected from the group comprising:
a) a tag;
b) a metabolic label;
c) a protein label;
d) an antibody to a tag, a metabolic label, or protein label;
e) an antibody to the A1 adenosine receptor protein or peptide;
f) an A1 adenosine receptor protein or peptide;
g) a protein or peptide in an A1 adenosine receptor signaling pathway;
h) an antibody to a protein or peptide in an A1 adenosine receptor signaling pathway;
i) an A1 adenosine receptor ligand;
j) a signaling molecule;
k) a molecule in an A1 adenosine receptor signaling pathway;
l) an antibody to a molecule in an A1 adenosine receptor signaling pathway; and
m) a molecule that can be measured in an A1 adenosine receptor signaling pathway.

A heterogeneous assay utilizes one reagent tagged with a fluorophore and thus is used to measure an analyte, such as LPS. This assay requires washing and filtering steps to separate the bound from the unbound labeled partner. These types of assays take advantage in many cases by the fluorescence properties of the rare earth elements in the lanthanide series. Commonly used lanthanides for use in these assays are samarium, europium, terbium, and dysprosium. These embodiments are utilized since they have large Stoke's shifts and extremely long emission half-lives when compared to other fluorophores. These types of assays tend to be competitive or binding type.

In TRF assays for the A1 adenosine receptor, fluorophores may be chelated, coupled, conjugated, or linked to a tag, such as S-nitroso-N-acetylpenacillamine (SNAP), Class II-associated invariant chain peptide (CLIP), chitin binding protein (CBP), maltose binding protein (MBP), or ACP/MCP tag, Green Fluorescent Protein (GFP), FLAG, glutathione-S-transferase (GST), histidine (HIS), acid azidohomoalanine (AHA) or HPG tags, or a metabolic label, such as a biomolecule with an azide or alkyne tag, inserted into the A1 adenosine receptor protein, G protein, dimers or subunits of G proteins, peptides for G proteins, dimers, or subunits of G proteins, other proteins involved in signaling pathway assays for the A1 adenosine receptor, such as a GTP binding assay, or peptides, oligonucleotides or epitopes for these proteins. Alternatively, in TRF assays, fluorophores may be chelated, conjugated, linked, or coupled to proteins or molecules in A1 adenosine receptor signaling pathways, including but not limited to the A1 adenosine receptor protein or peptide,a G protein, dimer or subunit of a G protein, or peptide for the G protein, dimer, or subunit of the G protein, other proteins, fusion proteins, peptides, oligonucleotides, or epitopes for these proteins, or molecules involved in signaling pathway assays for the A1 adenosine receptor, or tags inserted into or coupled to these proteins or molecules.

Alternatively, in TRF assays, fluorophores may be chelated, conjugated, linked, or coupled to antibodies to proteins or molecules in A1 adenosine receptor signaling pathways, including but not limited to the A1 adenosine receptor protein or peptide, a G protein, dimer, or subunit of a G protein, or peptide of a G protein, dimer, or subunit of a G protein, other proteins, fusion proteins, peptides, oligonucleotides, or epitopes for these proteins, or molecules involved in signaling pathway assays for the A1 adenosine receptor, or tags inserted into or coupled to these proteins or molecules.

Additional tags that can be used to link or chelate fluorophores for use in TRF assays include solubilization tags, thioredoxin and poly(NANP), which can be used for recombinant proteins expressed in bacteria. Other tags useful to link or chelate fluorophores for TRF assays include epitope tags, such as V5-tag, c-myc-tag, and HA tag. Additional tags for coupling, linking, or chelating fluorophores to proteins, peptides, molecules, epitopes, or oligonucleotides in TRF assays include isopeptag, biotin carboxyl-carrier protein (BCCP), calmodulin tag, nus-tag, S-tag, Softag 1, Softag 2, strep-tag, SBT-tag, and Ty-tag.

Alternatively, in TRF assays, fluorophores may be chelated, coupled, conjugated, or linked to other proteins, such as streptavidin, such as in LanthaScreen® products, which, because of its high affinity to biotin, may react with a protein, peptide, oligonucleotide, epitope tagged with biotin, an antibody to a protein, peptide, oligonucleotide, or epitope tagged with biotin, an antibody to a tag tagged with biotin, or other molecules or antibodies for these molecules involved in an A1 adenosine receptor binding or signaling pathway tagged with biotin. For example, in one such TRF assay, the fluorophore may be linked to streptavidin which in turn binds to a biotinylated peptide for a Gαi subunit protein. Alternatively, in TRF assays, fluorophores may be chelated, coupled, or linked to enzymes or substrates for enzymes commonly used in ELISAs and known to those skilled in the art of assay development. Some examples of enzymes include but are not limited to glycosidases, phosphatases, oxidases, peptidases, proteases, acetylcholinesterase, alkaline phosphatase, α-glycerophosphate, dehydrogenase, asparaginase, β-galactosidase, β-V-steroid isomerase, catalase, glucoamylase, glucose oxidase, glucose-6-phosphate dehydrogenase, horse radish peroxidase, malate dehydrogenase, ribonuclease, staphylococcal nuclease, triose phosphateisomerase, urease, and yeast alcohol dehydrogenase. Some examples of substrates for enzymes include but are not limited to tetramethyl benzene (TMB), o-phenylenediamine (OPD), coumarin substrates such as organic and inorganic esters and glycosides of 7-hydroxy-4-methylcoumarin (4-methylumbelliferone, 4-MU) and amides of 7-amino-4-methylcoumarin (AMC), fluorescein substrates, naphthyl substrates, substrates derived from resorufin, and the like. For example, in a TRF assay the fluorophore may be chelated to an antibody against a tag, such as a GST tag, inserted into a NF-κβ subunit, p65 recombinant protein. In another TRF assay, the anti-GST antibody chelated with a first binding partner fluorophore interacts with the GST tag, inserted into a NF-κβ subunit, p65 recombinant protein, which in turn interacts with a biotinylated NF-κβ specific dsDNA bound to streptavidin labeled with a second binding partner fluorophore.

Alternatively, in TRF assays, fluorophores may be chelated, conjugated, linked, coupled, or tagged to A1 adenosine receptor ligands, including antagonists and agonists, such as LPS, which bind to A1 adenosine receptors. Alternatively, in a TRF assay format, fluorophores may be chelated, coupled, or tagged to a signal transduction molecule, GTP, G proteins, dimers, or subunits of G proteins, peptides of G proteins or dimers or subunits of G proteins, proteins, such as interleukin-6 (IL-6) or a subunit for NF-κβ, or other signaling pathway molecules, such as cAMP or thromboxane, that can be measured following activation of the A1 adenosine receptor in signaling pathway assays for the A1 adenosine receptor. Alternatively, in a TRF assay format, fluorophores may be chelated or tagged to antibodies to proteins, such as IL-6 or a subunit for NF-κβ, or peptides, epitopes, or oligonucleotides for such proteins, tags inserted into such proteins, or to a signal transduction molecule, GTP, or other signaling pathway molecules, such as cAMP or thromboxane.

A list of A1 adenosine receptor ligands that can be coupled with fluorophores in A1 adenosine receptor TRF assays include, but are not limited to, agonists that activate the A1 adenosine receptor, such as N6 cyclopentyladenosine (CPA), 2-chloro-N6-cyclopentyladenosine (CCPA), 2-chloro-N⁶—[(R)-[(2-benzothiazolyl)thio]-2-propyl]-adenosine) (NNC-21-0136), 2′-O-methyl-N⁶-cyclohexyladenosine (SDZ WAG94), [1S-[1α,2β,3β4α((S*)]]-4-[7-[[1-[(3-chlorothien-2-yl)methyl]propyl]amino]-3H-imidazo[4,5-b]pyrid-3-yl]N-ethyl 2,3-dihydroxycyclopentanecarboxamide (AMP579), tecadenoson (2R,3S,4R)-2-(hydroxymethyl)-5-(6-((R)-tetrahydrofuran-3-ylamino)-9H-purin-9-yl)tetrahydrofuran-3,4-diol), selodenoson (2S,3S,4R)-5-(6-(cyclopentylamino)-9H-purin-9-yl)-N-ethyl-3,4-dihydroxytetrahydrofuran-2-carboxamide) and PJ-875, 2S,3S,4R)-2-((2-fluorophenylthio)methyl)-5-(6-((1R,2R)-2-hydroxycyclopentyl-amino)-9H-purin-9-yl)tetrahydrofuran-3,4-diol (CVT-3619), 3R,4S,5R)-2-(6-((1S,2S)-2-hydroxycyclopentylamino)-9H-purin-9-yl)-5-(hydroxymethyl)tetrahydrofuran-3,4-diol (GR 79236), 1S,2R,3R)-3-((trifluoromethoxy)methyl)-5-(6-(1-(5-(trifluromethyl)pyridine-2-yl)pyrrolidin-3-ylamino)-9H-purin-9-yl)cyclopentane-1,2-diol (ARA), capadenoson (2-amino-6-((2-(4-chlorophenyl)thiazol-4-yl)methylthio)-4-(4-(2-hydroxyethoxy)phenyl)pyridine-3,5-dicarbonitrile, Bay-68-4986), 2-amino-4,5,6,7-tetrahydrobenzo[b]thiophen-3-yl)(4-chlorophenyl)methanone (T-62) or LPS.

This list of A1 adenosine receptor ligands that can be coupled to fluorophores in A1 adenosine receptor TRF assays also includes, but is not limited to A1 adenosine receptor antagonists, such as 1,3 dipropyl-8-cyclopentyladenosine (DPCPX), 1,3-dipropyl-8-(2-(5,6-epoxy)norbornyl)xanthine (BG-9719), 3-[4-(2,6-dioxo-1,3-dipropyl-2,3,6,7-.tetrahydro-1H-purin-8-yl)-bicyclo[2.2.2]oct-1-yl]-propionic acid (BG-9928), 8-noradamantyl-1,3-dipropylxanthine (KW 3902), 3-[2-(4-aminophenyl)-ethyl]-8-benzyl-7-{2-ethyl-(2-hydroxy-ethyl)-amino]-ethyl}-1-propyl-3,7-dihydro-purine-2,6-dione (L-97-1), 4-(2-phenyl-7H-pyrrolo[2,3-d]pyrimidin-4-ylamino)cyclohexanol (SLV320), 3-(4-amino)phenethyl-1-propyl-8-cyclopentylxanthine (BW A844U), 8-cyclopentyl-3-(3-((4-fluorosulfonylbenzoyl)-oxy)propyl)-1-propylxanthine (FSCPX), bamiphylline, N6 endonorbornan-2-yl-9-methyladenine (N-0861), and C8-(N-methylisopropyl)-amino-N6-(5′-endohydroxy)-endonorbornan-2-yl-9-methyladenine (WRC-0571).

A1 adenosine receptor antagonists known in the art include, for example, those compounds described in U.S. Pat. Nos. 5,786,360, 6,489,332, 7,202, 252 B2, 7,247,639 B2, and in co-pending U.S. application Ser. No. 10/560,853, entitled “A₁ Adenosine Receptor Antagonists,” filed Jun. 7, 2004, Ser. No. 13/010,152, entitled “A1 Adenosine Receptor Diagnostic Probes,” filed Jan. 20, 2011, and PCT/US2008/087638, entitled “A₁ Adenosine Receptor Antagonists,” filed Dec. 19, 2008 all of which are herein incorporated by reference.

A list of A1 adenosine receptor signaling pathways suitable for development as A1 adenosine receptor TRF assays include, but are not limited to, GTP, adenylate cyclase, phospholipase C (PLC), phosphoinositide-3 kinase (PI3K), mitogen-activated protein kinases (MAPKs), extracellular receptor signal-induced kinase (ERK), phospholipase A2 (PLA₂), and protein kinase C (PKC).

Molecules associated with A1 adenosine receptor signaling pathways suitable for tagging with a fluorophore include, but are not limited to, G proteins, dimers, subunits, and peptides of G proteins, dimers, and subunits of G proteins, cAMP, NF-κB and subunits of NF-κB, IP3, DAG, interleukin-6 (IL-6), p38, heat shock protein, thromboxane, matrix metaloproteinanses, and growth factors. Moreover, other effectors associated with A1 adenosine receptors signaling pathways suitable for tagging with fluorophores include proteins for potassium channels and calcium channels and ions such as potassium and calcium.

Homogeneous TRF assays, such as TR-FRET technologies, involve donor and acceptor fluorophore pairings, that is a plurality of fluorophores, and involves both ligand binding and functional assays for GPCRs, such as the A1 adenosine receptor, in an homogeneous assay format that does not require wash and filter or wash steps of the heterogeneous assay, with low background noise, and with high sensitivity and specificity. In homogeneous TR-FRET (HTRF) assays, two labeled partners with fluorophores are required for energy transfer. This energy transfer occurs only when the two molecules are in direct proximity to one another. The first fluorophore acts as an energy donor and second fluorophore acts as an energy acceptor. The efficiency of the energy transfer is a function of the distance between the long-lived fluorescence donor and the short-lived fluorescence acceptor dyes. The most commonly used donor lanthanides used in TR-FRET assays are europium and terbium. Other donor lanthanides include samarium and dysprosium. There are a number of resonance energy acceptors including, XL665 (allophycocyanin), d2, phycobiliprotein, tetramethylrhodamine, fluorescein, thionine, R phycocyanin, phycoerythrocyanine, C phycoerythrin, and others. Further examples suitable for use in the present invention can be found in U.S. Pat. No. 6,908,769 incorporated herein by reference in its entirety. The most commonly used energy transfer acceptors in HTRF assays are d2 or XL665. A typical donor/acceptor pair in an HTRF assay that produces high FRET efficiency at excitation of 337 nm is europium cryptate as the donor with an emission of 620 nm and XL665 as the acceptor with an emission of 665 nm. The natural short-lived fluorescent emission of the free acceptor, XL665, compared to the long-lived emission in the energy transfer process (due to the long-lived fluorescent lifetime of europium cryptate as the donor) allows a clear distinction between bound (which occurs during energy transfer when the molecule tagged with XL665 comes into close proximity to the molecule tagged with europium cryptate) and free XL665.

There are a number of advantages associated with HTRF/TR-FRET assays, including homogeneous assay format, rapid, high sensitivity and specificity, low background noise, robustness with little interference from medium background, such as plasma, suitable for use with GPCRs expressed in membranes, tolerant of divalent ions such as Mg²⁺ or other assay additives, such as DMSO and EDTA, and assay flexibility, such as adaptable to high throughput screening, automated liquid handling, and miniaturization. Moreover, HTRF assays are easy to perform once developed and determined to work with particular GPCRs and proteins and thus, are user friendly and results are available usually within 2 hours.

Examples of HTRF, TR-FRET assays can be found in U.S. Pat. Nos. 7,674,584, 5,998,146, 5,512,493, 5,527,684, 6,352,672, 5,220,012, 5,432,101, 5,457,185, 5,534,622, 5,346,996, 5,162,508, 5,512,493, 5,627,074, 5,527,684, 6,515,113, 6,864,103, 7,442,558, 6,406,297, 7,018,850, 7,404,912, and US patent applications US20040115130, US20060024775, US 20060292651, US20070243568, US20070207532 incorporated herein by reference in their entirety.

A protein source for the A1 adenosine receptor can be provided in any form compatible with the assay of the present invention. The A1 adenosine receptor may be a recombinant protein stably transfected into cells as, including but not limited to, plants, yeast, CHO, or Sf9 cells. Membranes from these cells, or the like, can be utilized for providing the source for the A1 adenosine receptor protein for the TRF assay. Other sources for the A1 adenosine receptor protein include other cell types, tissues, plants, bacteria, yeast, and solubilized or purified A1 adenosine receptor protein isolated from a cell, tissue, plants, bacteria, or membranes stably expressing the A1 adenosine receptor protein, or recombinant A1 adenosine receptor protein. One skilled in the art of assays could easily determine and provide the A1 adenosine receptor protein in an acceptable form.

In the running of the assay, the selected TRF assay, heterogeneous or homogeneous, is set up using protocols from the manufacturer of the tests, as well as buffer and solution characteristics, which must be determined by a skilled user of these assays using the A1 adenosine receptor and LPS. Once developed a test sample is introduced into the assay for a quantitative determination of the amount of LPS present in the sample. That result can be determined from a standard curve for LPS generated with samples containing known amounts of LPS in the assay. The determination of the presence and amount or level of LPS in the sample can be used for the diagnosis of sepsis, Gram-negative infection, or LPS-related conditions in the animal. The present invention also provides a kit for the detection and measurement of LPS in a sample and methods of use for the diagnosis of sepsis, Gram-negative infection, or LPS-related conditions in an animal. Kits are provided for measuring LPS levels in a sample. These kits include a protein source for the A1 adenosine receptor, at least one fluorophore, buffers specific for the A1 adenosine receptor TRF assay and LPS, as well as LPS standards. In one embodiment the kit provides for a GTP-lanthanide, such as GTP chelated to europium (Eu-GTP) as a fluorophore for performing an A1 adenosine receptor TRF GTP binding assay.

The following non-limiting examples are provided to further illustrate embodiments of the present invention.

### EXAMPLE 1

Preparation of Membranes from CHO Cells Expressing the Recombinant rat A1 Adenosine Receptor
1. Collect CHO cells from suspension by centrifuging at 170×g for 10 minutes.
2. Wash cells twice with one volume (50 mL) of PBS.

### **All Remaining Steps Carried Out on Ice or at 4° C.**

3. Resuspend pellets in cold Buffer A (5 mL per 50 mL of original culture volume).
4. Homogenize the cells in a Polytron (Brinkmann) for 20 seconds.
5. Centrifuge the homogenate at 1000×g at 4C for 10 minutes.
6. Collect supernatant and centrifuge at 30,000×g for 30 minutes.
7. Discard supernatant and reserve pellet.
8. Wash pellet twice with Buffer A.
9. Gently resuspend pellet by pipetting in Buffer B to achieve a final concentration of 2 mg protein/mL
10. Aliquot the membrane suspension in units of 25 uL and snap freeze.
11. Store aliquots of membrane suspension at −80° C.

| Buffer A | Buffer B |
|---|---|
| 10 mM HEPES, pH 7.4 | 10 mM HEPES, pH 7.4 |
| 20 mM Na2EDTA | 1 mM Na2EDTA |
| 20 μg/mL benzamidine | 10% sucrose |
| 2 μg/ml leupeptin | 20 μg/mL benzamidine |
| 2 μg/mL aprotinin | 2 μg/ml leupeptin |
| 2 μg/mL pepstatin | 2 μg/mL aprotinin |
| 100 μM phenylmethylsulfonyl | 2 μg/mL pepstatin |
| fluoride | 100 μM phenylmethylsulfonyl fluoride |

### EXAMPLE 2

Saturation Binding Studies to Determine Affinity of [3H]1,3 dipropyl-8-cyclopentylxanthine (DPCPX) for Recombinant Rat A1 Adenosine Receptor Expressed in CHO Cells.

For saturation binding assays, membranes prepared from CHO cells stably transfected with the recombinant rat A1 adenosine receptor (5-20 μg protein/well) are incubated with increasing concentrations of [3H]-DPCPX (Perkin Elmer, Cambridge, Mass.) (0.01 nM-10 nM) in a final assay volume of 200 μL. For each concentration of [3H]-DPCPX, total binding and nonspecific binding are determined in triplicate. Total binding is defined in the absence of a competing ligand and nonspecific binding is determined in presence of DPCPX (Sigma-Aldrich, St. Louis, Mo.) (10 μM). The DPCPX stock solution is prepared in DMSO (final concentration of DMSO in the assay is 0.01%). Assay buffer consists of 50 mM Tris HCl (pH 7.4), 10 mM MgCl₂, and adenosine deaminase (Sigma-Aldrich) (0.2 units/mL). All assay components are added to a polypropylene, deep well plate (Thermo Fisher Scientific, Waltham, Mass.) and then the plate is gently agitated to mix the components. Assay is performed using sterile technique, sterile reagents, and sterile consumables. Membranes are incubated for 60 minutes at 25° C. then the assay is terminated by rapid filtration through a GF/B filter mat (Perkin Elmer) using an automated vacuum manifold (Mach III, Tomtec, Hamden, Conn.). Each well is rapidly washed four times with 300 μL of ice-cold wash buffer (Tris-HCl [50 mM, pH 7.4] and MgCl₂ [10 mM]). The filter mat is dried, embedded with solid scintillant (PerkinElmer), and counted for 3H using a scintillation counter (1450 Microbeta, PerkinElmer). An aliquot of the membrane sample diluted for the assay is used to quantitate total protein using the BCA assay (Thermo Fisher Scientific) using BSA as a standard. For each concentration of [3H]-DPCPX, data are expressed as specific CPM bound (the difference between total CPM bound and nonspecific CPM bound) and then converted to fmol/mg protein. Data are plotted as specific bound (fmol/mg protein) versus concentration of [3H]-DPCPX then analyzed by nonlinear regression (curve fit), using the one-site model to determine K_{D} and Bₘₐₓ (GraphPad Prism, version 5.01, La Jolla, Calif.). The final data from a minimum of three independent experiments are expressed as the mean±SEM.

### EXAMPLE 3

Competition Binding Studies to Determine Affinity (Ki) of BW A844U-Eu for Recombinant Rat A1 Adenosine Receptor Expressed in Membranes from CHO Cells Labeled with [3H]-DPCPX

For competition binding assays, membranes prepared from CHO cells stably transfected with the recombinant rat A1 adenosine receptor (5-20 μg protein/well) are incubated with [3H]-DPCPX (at K_{D}, as defined in saturation binding studies) in a final assay volume of 200 μL. Total binding is defined in the absence of a competing ligand and nonspecific binding is determined in presence of DPCPX (10 μM). Assay buffer consists of 50 mM Tris HCl (pH 7.4), 10 mM MgCl₂, and adenosine deaminase (0.2 units/mL). BW A844U coupled to europium (BW A844U-Eu) is evaluated at concentrations ranging from 0.01 nM-10 μM. BW A844U-Eu stock solution is prepared in DMSO (final concentration of DMSO in the assay is 0.01%). Control ligands are CPA (N⁶-cyclopentyladenosine) (Sigma-Aldrich, St. Louis, Mo.) (0.01 nM-10 μM) and DPCPX (0.01 nM-10 μM). The stock solutions of DPCPX and CPA are prepared in DMSO (final concentration of DMSO in the assay is 0.01%). Each assay point is evaluated in triplicate. All assay components are added to a polypropylene, deep well plate (Thermo Fisher Scientific) and the plate is gently agitated to mix the components. Assay is performed using sterile technique, sterile reagents, and sterile consumables. Membranes are incubated for 60 minutes at 25° C. then the assay is terminated by rapid filtration through a GF/B filter mat (PerkinElmer) using a vacuum manifold (Mach III, Tomtec, Hamden, Conn.). Each well is rapidly washed four times with 300 μL of ice-cold wash buffer (Tris-HCl [50 mM, pH 7.4] and MgCl₂ [10 mM]). Filter mat is dried, embedded with solid scintillant (PerkinElmer), and then counted for 3H using a scintillation counter (1450 Microbeta, PerkinElmer). For each concentration of test ligand, percent specific bound is calculated as [(bound—nonspecific bound)/(total bound—nonspecific bound)]*100. Data are plotted as percent specific bound versus log concentration of competing ligand. The data are analyzed by nonlinear regression (curve fit) using a competitive binding model to determine Ki (GraphPad Prism, version 5.01). The final data from a minimum of three independent experiments are expressed as the mean±SEM.

### EXAMPLE 4

Time Resolved Fluorescence (TRF) Saturation Binding Studies to Determine Affinity (K_{D}) for BW A844U-Eu for Recombinant Rat A1 Adenosine Receptor Expressed in Membranes from CHO Cells

For TRF saturation binding assays, membranes prepared from CHO cells stably transfected with the recombinant rat A1 adenosine receptor (5-20 μg protein/well) are incubated with increasing concentrations of BW A844U-Eu (0.01 nM-1 μM) in a final assay volume of 200 μL. BW A844U-Eu stock solution is prepared in DMSO (final concentration of DMSO in the assay is 0.01%). For each concentration of BW A844U-Eu, total binding and nonspecific binding are determined. Total binding is defined in the absence of a competing ligand. Nonspecific binding is determined in presence of N⁶-R-phenylisopropyladenosine (R-PIA, Sigma-Aldrich) (100 μM). The R-PIA stock solution is prepared in endotoxin-free water. Assay buffer consists of 50 mM Tris HCl (pH 7.4), 10 mM MgCl₂, and adenosine deaminase (0.2 units/mL). Each assay point is evaluated in triplicate. All assay components are added to an Acrowell™ 96-well filter plate (Pall Life Sciences, Ann Arbor, Mich.) and the plate is gently agitated to mix the components. Assay is performed using sterile technique, sterile reagents, and sterile consumables. Membranes are incubated for 60 minutes at 25° C. then the assay is terminated by rapid filtration through the BioTrace polyvinylidene fluoride filter of the Acrowell™ filter plate using a vacuum manifold (Pall Life Sciences). Each well is rapidly washed four times with 300 μL of ice-cold wash buffer (Tris-HCl [50 mM, pH 7.4] and MgCl₂ [10 mM]). Fluorescence in each well is measured with excitation of 320 nm and emission of 620 nm on a microplate reader with TRF capability (Infinite F-200 PRO, Tecan, Grodig, Austria). An aliquot of the membrane sample diluted for the assay is used to quantitate total protein using the BCA assay (Thermo Fisher Scientific) using BSA as a standard. For each concentration of BW A844U-Eu, data are expressed as specific bound (the difference between total bound and nonspecific bound) and then converted to fmol/mg protein. Data are plotted as specific bound (fmol/mg protein) versus concentration of BWA844U-Eu and then analyzed by nonlinear regression (curve fit), using the one-site model, to determine K_{D} and Bₘₐₓ (GraphPad Prism, version 5.01). The final data from at least three independent experiments are expressed as the mean±SEM.

### EXAMPLE 5

### A1 Adenosine Receptor TRF Competition Binding Assay for Lipopolysaccharide (LPS)/Endotoxin

For competition binding assays, membranes prepared from CHO cells stably transfected with the recombinant rat A1 adenosine receptor (5-20 μg protein/well) are incubated with BW A844U-Eu at K_{D} as determined by saturation binding studies above. Total binding is defined in the absence of a competing ligand. Nonspecific binding is determined in presence of N⁶-R-phenylisopropyladenosine (R-PIA) (100 μM). The R-PIA stock solution is prepared in endotoxin-free water. Assay buffer consists of 50 mM Tris HCl (pH 7.4), 10 mM MgCl₂, and adenosine deaminase (0.2 units/mL). Test ligands are LPS/endotoxin (USP, Rockville, Md.) (0.01-750 ng/mL) [or CPA) (0.01 nM-1 μM) as a positive control]. The LPS stock solution is prepared by dissolving 10,000 endotoxin units (corresponding to 1000 ng) in 333 μL of endotoxin-free water. The CPA stock solution is prepared in DMSO (final concentration of DMSO in the assay is 0.01%). Final assay volume is 200 μL. Each assay point is evaluated in triplicate. All assay components are added to an Acrowell™ 96-well filter plate (Pall Life Sciences) then the plate is gently agitated to mix the components. Assay is performed using sterile technique, sterile reagents, and sterile consumables. Assay components are incubated for 60 minutes at 25° C. then the assay is terminated by rapid filtration through the BioTrace polyvinylidene fluoride filter of the Acrowell™ filter plate using a vacuum manifold (Pall Life Sciences). Each well is rapidly washed two-four times with 300 μL of ice-cold wash buffer (Tris-HCl [50 mM, pH 7.4] and MgCl₂ [10 mM]). Fluorescence in each well is measured with excitation of 320 nm and emission of 620 nm on a microplate reader with TRF capability (Infinite F-200 PRO, Tecan, Grodig, Austria). For each concentration of test ligand, percent specific bound is calculated as [(bound−nonspecific bound)/(total bound−nonspecific bound)]*100. Data are plotted as percent specific bound versus concentration of competing ligand [the log molar (CPA) and log g/mL (LPS)], then analyzed by nonlinear regression (curve fit) using a competitive binding model to determine Ki (GraphPad Prism, version 5.01). The final data from a minimum of three independent assays are expressed as the mean±SEM.

### EXAMPLE 6

A1 Adenosine Receptor Saturation Binding Studies to Measure Effect of the Polyclonal Antibody for Recombinant Rat A1 Adenosine Receptor Tagged with an Acceptor Fluorophore, U-Light, on the Binding of [3H] DPCPX.

Membranes prepared from CHO cells stably transfected with the recombinant rat A1 adenosine receptor (5-20 μg protein/well) are incubated with increasing concentrations of [3H]-DPCPX (PerkinElmer, Cambridge, Mass.) (0.01 nM-10 nM) in the presence or absence of a polyclonal antibody for the recombinant rat A1 adenosine receptor tagged with an acceptor fluorophore, ULight (Perkin Elmer) (1:10-1:10,000). For each concentration of [3H]-DPCPX with and without antibody, total binding and nonspecific binding are determined. Total binding is defined in the absence of a competing ligand. Nonspecific binding is determined in presence of DPCPX (Sigma-Aldrich, St. Louis, Mo.) (10 μM). The DPCPX stock solution is prepared in DMSO (final concentration of DMSO in the assay is 0.01%). Assay buffer consists of 50 mM Tris HCl (pH 7.4), 10 mM MgCl₂, and adenosine deaminase (Sigma-Aldrich) (0.2 units/mL). Total assay volume is 200 uL. Each assay point is evaluated in triplicate. All assay components are added to a polypropylene, deep well plate (Thermo Fisher Scientific, Waltham, Mass.) and then the plate is gently agitated to mix the components. Assay is performed using sterile technique, sterile reagents, and sterile consumables. Membranes are incubated for 60 minutes at 25° C. then the assay is terminated by rapid filtration through a GF/B filter mat (PerkinElmer) using an automated vacuum manifold (Mach III, Tomtec, Hamden, Conn.). Each well is rapidly washed four times with 300 μL of ice-cold wash buffer (Tris-HCl [50 mM, pH 7.4] and MgCl₂ [10 mM]). The filter mat is dried, embedded with solid scintillant (PerkinElmer), and counted for 3H using a scintillation counter (1450 Microbeta, PerkinElmer). An aliquot of the membrane sample diluted for the assay is used to quantitate total protein using the BCA assay (Thermo Fisher Scientific) using BSA as a standard. Data are expressed as specific CPM bound ( the difference between total CPM bound and nonspecific CPM bound), plotted as specific bound (fmol/mg protein) versus concentration of [3H]-DPCPX, and then analyzed by nonlinear regression (curve fit) using the one-site model to determine K_{D} and Bₘₐₓ (GraphPad Prism, version 5.01, La Jolla, Calif.). The final data (K_{D} and Bₘₐₓ) from a minimum of three independent experiments are expressed as the mean±SEM. To evaluate the effect of the polyclonal antibody for the recombinant rat A1 adenosine receptor tagged with an acceptor fluorophore, ULight on the A1 adenosine receptor ligand recognition site, Student's t-test for unpaired data will be used to compare the K_{D} and Bₘₐₓ of [3H]-DPCPX in the absence and presence of antibody. P-value<0.05 is considered significantly different.

### EXAMPLE 7

### A1 Adenosine Receptor Homogeneous Time Resolved Fluorescence (HTRF) Competition Assay to Generate Standard Curve for Lipopolysaccharide (LPS)/Endotoxin and to Measure Level of LPS in Test Sample

To generate a standard LPS/endotoxin standard curve, CHO cell membranes expressing the recombinant rat A1 adenosine receptor (5-20 μg protein/well) are incubated in the presence of a polyclonal antibody for recombinant rat A1 adenosine receptor tagged with an acceptor fluorophore, ULight (Perkin Elmer) (1:10-1:10,000) and BW A844U-Eu (0.5-20 nM) (ca. K_{D} as determined by saturation binding studies above) in Greiner white 96 well plates (Greiner Bio-One North America, Monroe, N.C.). Total binding is defined in the absence of test ligand and nonspecific binding is defined by the addition of R-PIA (100 μM). The activating ligand, LPS/endotoxin (USP, Rockville, Md.), is evaluated at 8 concentrations ranging from 0.01-750 ng/mL. The LPS/endotoxin stock solution is prepared by dissolving 10,000 endotoxin units (corresponding to 1000 ng) in 333 μL of endotoxin-free water. Additional assay components include assay buffer (Tris-HCl [50 mM, pH 7.4], MgCl₂ [10 mM], and adenosine deaminase (0.2 units/mL). The total assay volume is 200 μL. Each assay point is evaluated in triplicate. All assay components are added to the plate and then the plate is gently agitated to mix the components. Assay is performed using sterile techniques, sterile reagents, and sterile consumables. Membranes are incubated for 60 minutes at 25° C. Following the incubation, fluorescence is measured with excitation of 320 nm and emissions at 665 and 620 nm on a microplate reader with HTRF capability (Infinite F-200 PRO, Tecan, Grodig, Austria). Data for total, nonspecific, and LPS are expressed as the ratio of the measurements at 665 and 620 (665/620). Basal activity is then calculated as the difference between total and nonspecific ratio measurements. Percent (%) basal activity for LPS is calculated as follows: [(test condition−nonspecific)/(basal activity)]*100. The concentration-response curve for LPS is plotted as a function of % basal activity versus log concentration of LPS (g/mL). This curve is analyzed by nonlinear regression (GraphPad Prism, version 5.04, GraphPad Software, La Jolla, Calif.) using a sigmoidal dose-response curve with variable slope to determine EC50. Each standard curve represents a minimum of three independent experiments and final data are expressed as the mean±SEM.

To measure LPS/endotoxin levels in test samples, CHO cell membranes expressing the recombinant rat A1 adenosine receptor (5-20 μg protein/well) are incubated in the presence of a polyclonal antibody for rat A1 adenosine receptor tagged with an acceptor fluorophore, ULight (Perkin Elmer) (1:10-1:10,000) and BW A844U-Eu (0.5-20 nM) (ca. K_{D} as determined by saturation binding studies above) and test samples or positive controls with LPS (10 pg/mL 1 ng/mL, 10 ng/mL, and 100 ng/mL) in Greiner white 96 well plates (Greiner Bio-One North America, Monroe, N.C.). Total binding is defined in the absence of a competing ligand. Nonspecific binding is determined in presence of N⁶-R-phenylisopropyladenosine (R-PIA) (100 μM). The R-PIA stock solution is prepared in endotoxin-free water. For positive LPS controls the endotoxin stock solution is prepared by dissolving 10,000 endotoxin units (corresponding to 1000 ng) in 333 μL of endotoxin free water. Additional assay components include assay buffer (Tris-HCl [50 mM, pH 7.4], MgCl₂ [10 mM], and adenosine deaminase (0.2 units/mL). The total assay volume is 200 μL. Each assay point is evaluated in triplicate. All assay components are added to the plate and then the plate is gently agitated to mix the components. Assay is performed using sterile technique, sterile reagents, and sterile consumables. Membranes are incubated for 60 minutes at 25° C. Following the incubation, fluorescence is measured with excitation of 320 nm and emissions at 665 and 620 nm on a microplate reader with HTRF capability (Infinite F-200 PRO, Tecan, Grodig, Austria). Data for total, nonspecific, test samples, and positive LPS controls are expressed as the ratio of the measurements at 665 and 620 (665/620). Basal activity is then calculated as the difference between total and nonspecific ratio measurements. Percent (%) basal activity for test sample or positive LPS control is calculated as follows: [(test condition−nonspecific)/(basal activity)]*100. Level of LPS in the test sample or positive control is determined from the standard curve for LPS by comparing the fluorescence measurement expressed in units of % basal activity for the sample or positive control to % basal activity measurements for samples spiked with known concentrations of LPS to generate the standard curve for LPS in the HTRF competition assay described above.

### EXAMPLE 8

### A1 Adenosine Receptor TRF Eu-GTP Binding Assay to Generate Standard Curve for Lipopolysaccharide (LPS)/Endotoxin and to Measure Level of LPS in Test Sample

To generate a standard LPS/endotoxin standard curve membranes prepared from CHO cells (1 unit/well corresponding to approximately 20 μg of protein per well; PerkinElmer, Waltham, Mass.) expressing the recombinant rat A1 adenosine receptor are preincubated in an Acrowell filter plate (Pall Life Sciences, Ann Arbor, Mich.) in the presence of LPS/endotoxin (USP, Rockville, Md.) (0.01-750 ng/mL). The endotoxin stock solution is prepared by dissolving 10,000 endotoxin units (corresponding to 1000 ng) in 333 μL of endotoxin free water. Additional assay components include assay buffer (Tris-HCl [50 mM, pH 7.4], MgCl₂ [10 mM], NaCl [100 mM]), GDP (10 μM), saponin (125 μg/mL), and adenosine deaminase (0.2 units/mL). All reagents and buffers are prepared using endotoxin-free water. Total volume for the 60 min preincubation is 150 μL. Following the preincubation at 25° C., 50 μL of Eu-GTP (PerkinElmer) is added to achieve a final Eu-GTP concentration of 10 nM in a total volume of 200 μL. Each assay point is evaluated in triplicate. Total binding is defined in the absence of LPS and nonspecific binding is defined by the addition of GTPγS (10 μM). Assay is performed using sterile technique, sterile reagents, and sterile consumables. Following a 30 minute incubation at 25° C., the assay is terminated by filtering using a vacuum manifold (Pall Life Sciences, Ann Arbor, Mich.). Membranes are captured on the BioTrace polyvinylidene fluoride filter of the filter plate. Each well is washed two-four times with 300 μL of wash buffer (Tris-HCl [50 mM, pH 7.4] and MgCl₂ [10 mM]). Fluorescence in each well is determined with an Infinite F-200 PRO (Tecan, Grodig, Austria) using fluorescent top read with an excitation wavelength of 320 nm, emission wavelength of 620 nm, and gain setting of 109. Eu-GTP basal activity is calculated as the difference between total Eu-GTP bound and nonspecific Eu-GTP bound. Percent (%) basal is calculated as follows: [(test condition−nonspecific)/(basal activity)]*100. The concentration-response standard curve for LPS is plotted as a function of Eu-GTP % basal activity versus log concentration of LPS (g/mL). The standard curve is analyzed by nonlinear regression (GraphPad Prism, version 5, GraphPad Software, La Jolla, Calif.) using a sigmoidal dose-response curve with variable slope to determine EC50. Each standard curve represents a minimum of three independent experiments and final data are expressed as the mean±SEM.

To measure LPS/endotoxin levels in test samples, membranes prepared from CHO cells (1 unit/well corresponding to approximately 20 μg of protein per well; PerkinElmer, Waltham, Mass.) expressing the recombinant rat A1 adenosine receptor are preincubated in an Acrowell filter plate (Pall Life Sciences, Ann Arbor, Mich.) in the presence of a test sample or positive control with LPS/endotoxin (USP, Rockville, Md.) (10 pg/mL 1 ng/mL, 10 ng/mL, and 100 ng/mL)). For positive LPS controls the endotoxin stock solution is prepared by dissolving 10,000 endotoxin units (corresponding to 1000 ng) in 333 μL of endotoxin free water. Additional assay components include assay buffer (Tris-HCl [50 mM, pH 7.4], MgCl₂ [10 mM], NaCl [100 mM]), GDP (10 μM), saponin (125 μg/mL), and adenosine deaminase (0.2 units/mL). All reagents and buffers are prepared using endotoxin-free water. Total volume for the 60 minutes preincubation is 150 μL. Following the preincubation at 25° C., 50 μL of Eu-GTP (Perkin Elmer) is added to achieve a final Eu-GTP concentration of 10 nM in a total volume of 200 μL. Each assay point is evaluated in triplicate. Total binding is defined in the absence of LPS and nonspecific binding is defined by the addition of GTPγS (10 μM). Assay is performed using sterile technique, sterile reagents, and sterile consumables. Following a 30 minute incubation at 25° C., the assay is terminated by filtering using a vacuum manifold (Pall Life Sciences, Ann Arbor, Mich.). Membranes are captured on the BioTrace polyvinylidene fluoride filter of the filter plate. Each well is washed two-four times with 300 μL of wash buffer (Tris-HCl [50 mM, pH 7.4] and MgCl₂ [10 mM]). Fluorescence in each well is determined with an Infinite F-200 PRO (Tecan, Grodig, Austria) using fluorescent top read with an excitation wavelength of 320 nm, emission wavelength of 620 nm, and gain setting of 109. Eu-GTP basal activity is calculated as the difference between total Eu-GTP bound and nonspecific Eu-GTP bound. Percent (%) basal activity is calculated as follows: [(test condition−nonspecific)/(basal activity)]*100. Level of LPS in the test sample or positive control is determined from the standard curve for LPS by comparing the fluorescence measurement expressed in units of % basal activity for the sample or positive control to % basal activity measurements for samples spiked with known concentrations of LPS to generate the standard curve for LPS in the TRF Eu-GTP binding assay described above.

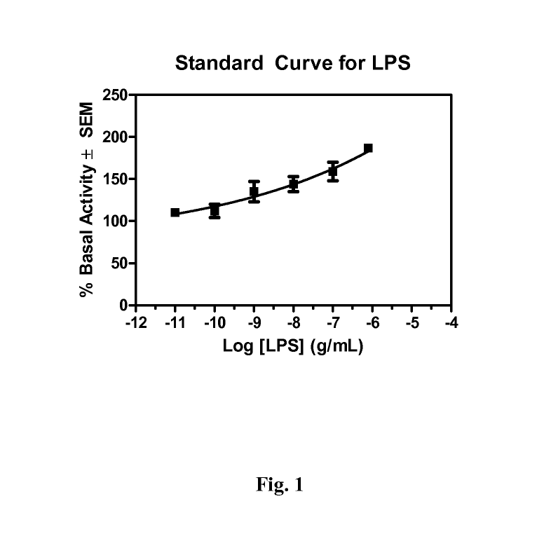

The FIG. 1 shows a concentration-response standard curve for LPS from a TRF Eu-GTP binding assay plotted as a function of % basal activity versus log concentration of LPS, (g/mL). Details for the TRF Eu-GTP binding assay are described in Example 8. This standard curve was analyzed by nonlinear regression (GraphPad Prism, version 5, GraphPad Software, La Jolla, Calif.) using a sigmoidal dose-response curve with variable slope to determine EC50. The standard curve represents the mean of 8-10 independent assays for concentrations of LPS, (10 pg/mL−100 ng/mL). The sensitivity of this assay is 10 pg/mL.

### EXAMPLE 9

### A1 Adenosine Receptor HTRF GTP Binding Assay to Generate Standard Curve for Lipopolysaccharide (LPS)/Endotoxin and to Measure Level of LPS in Test Sample.

To generate a standard LPS/endotoxin standard curve, CHO cell membranes expressing the recombinant rat A1 adenosine receptor (Invitrogen) (5-20 μg protein/well) are incubated with LPS/endotoxin (USP, Rockville, Md.) (0.01-750 ng/mL) in Greiner white microtiter 96 well plates. The LPS stock solution is prepared by dissolving 10,000 endotoxin units (corresponding to 1000 ng) in 333 μL of endotoxin free water. Total binding is defined in the absence LPS and nonspecific binding is defined by the addition of GTPγS (10 μM). Additional assay components include assay buffer (Tris-HCl [50 mM, pH 7.4], MgCl₂ [10 mM], NaCl [100 mM]), GDP (10 μM), saponin (125 μg/mL), and adenosine deaminase (0.2 units/mL). Following an incubation for 30-60 minutes at 25° C. a polyclonal antibody for recombinant rat A1 adenosine receptor tagged with ULight (Perkin Elmer) (1:10-1:10,000) and Eu-GTP (Perkin Elmer) (5-20 nM) are added to the assay. The total assay volume is 200 μl. Each assay point is evaluated in triplicate. Assay is performed using sterile technique, sterile reagents, and sterile consumables. Following an additional 30-60 minutes incubation fluorescence is measured with excitation of 320 nm and emissions at 665 and 620 nm on a Tecan Infinite F-200 PRO (Tecan, Grodig, Austria). Data for total, nonspecific, and LPS are expressed as the ratio of the measurements at 665 and 620 (665/620). Eu-GTP basal activity is then calculated as the difference between total and nonspecific ratio measurements. Percent (%) basal activity for LPS is calculated as follows: [(test condition−nonspecific)/(basal activity)]*100. The concentration-response curve for LPS is plotted as a function of % basal activity versus log concentration of LPS (g/mL). This curve is analyzed by nonlinear regression (GraphPad Prism, version 5.04, GraphPad Software, La Jolla, Calif.) using a sigmoidal dose-response curve with variable slope to determine EC50. Each standard curve represents a minimum of three independent experiments and final data are expressed as the mean±SEM.

To measure LPS/endotoxin levels in test samples, CHO cell membranes expressing the recombinant rat A1 adenosine receptor (Invitrogen) (5-20 μg protein/well) are incubated with a test sample or positive control with LPS/endotoxin (USP, Rockville, Md.) (10 pg/mL 1 ng/mL, 10 ng/mL, and 100 ng/mL)) in Greiner white microtiter 96 well plates. For positive LPS controls the endotoxin stock solution is prepared by dissolving 10,000 endotoxin units (corresponding to 1000 ng) in 333 μL of endotoxin free water. Total binding is defined in the absence LPS and nonspecific binding is defined by the addition of GTPγS (10 μM). Additional assay components include assay buffer (Tris-HCl [50 mM, pH 7.4], MgCl₂ [10 mM], NaCl [100 mM]), GDP (10 μM), saponin (125 μg/mL), and adenosine deaminase (0.2 units/mL). Following an incubation for 30-60 minutes at 25° C. a polyclonal antibody for recombinant rat A1 adenosine receptor tagged with ULight (Perkin Elmer) (1:10-1:10,000) and Eu-GTP (Perkin Elmer) (5-20 nM) are added to the assay. The total assay volume is 200 μl. Each assay point is evaluated in triplicate. Assay is performed using sterile technique, sterile reagents, and sterile consumables. Following an additional 30-60 minutes incubation fluorescence is measured with excitation of 320 nm and emissions at 665 and 620 nm on a Tecan Infinite F-200 PRO (Tecan, Grodig, Austria). Data for total, nonspecific, test samples, and positive LPS controls are expressed as the ratio of the measurements at 665 and 620 (665/620). Eu-GTP basal activity is then calculated as the difference between total and nonspecific ratio measurements. Percent (%) basal activity for test sample or positive LPS control is calculated as follows: [(test condition−nonspecific)/(basal activity)]*100. Level of LPS in the test sample or positive control is determined from the standard curve for LPS by comparing the fluorescence measurement expressed in units of % basal activity for the sample or positive control to % basal activity measurements for samples spiked with known concentrations of LPS to generate the standard curve for LPS in the HTRF GTP binding assay described above.

### EXAMPLE 10

Sensitivity and Specificity for an A1 Adenosine Receptor HTRF GTP Binding Assay Compared to the Sensitivity and Specificity of the Endotoxin Assay Activity (EAA™) Assay to Measure LPS in Blood of Rats with Cecal Ligation and Puncture (CLP) Induced Sepsis

### Rat Model of CLP-Induced Sepsis: Surgical Procedures and Daily Monitoring of Animals

All surgical procedures and animal care are in accordance with NIH guidelines and are approved by the Animal Care Committee. Male pathogen-free Sprague-Dawley rats (275-325 g, Harlan, Indianapolis, Ind.) are housed in positive-pressure isolation carrels with free access to food and water before experiments and in a BSL-2 biohazard laboratory thereafter. Under general anesthesia (3% isoflurane, 97% O₂) and using aseptic technique throughout, a 2-cm incision is made in the midventral neck to expose and catheterize the left carotid artery and right jugular vein (PE-50; Clay Adams, Parsippany, N.J.). Baseline vital signs (rectal temperature, pulsatile and mean arterial blood pressure, carotid pulse rate, respiratory frequency, degree of piloerection, presence or periorbital bleeding or nasal discharge) are determined and an initial arterial blood sample is obtained (1.0 mL) for hematology, blood gases, plasma sample, and culture. Withdrawn blood is replaced with 3× the volume of sterile 0.9% NaCl (normal saline, NS). The surgical incision is closed, anesthesia is withdrawn, and the animal is monitored until conscious (typically 3-5 min) and then returned to its cage. Catheters are shielded within stainless steel springs and mounted to swivels (Instech) fastened to the cage lid to allow free movement and access to food and water. The next morning (18-24 h later), animals are re-anesthetized and a sterile 4 cm midventral abdominal incision is made to expose the intestines. The intestines are gently retracted below the ileocecal valve with 3-0 silk and two perforations are then made in the cecum by its “through and through” penetration with a 19-gauge needle on both its mesenteric and anti-mesenteric surfaces. The perforated cecum is gently squeezed until at least 50 μL of feces extrude onto each of the penetrated surfaces, and then the bowel is gently reinserted into the abdomen and the incision closed. A single post-operative NS bolus is provided (50 mL/kg s.c.) for fluid support. Monitoring of animals for hemodynamics is recorded at least at 0, 3, 6, 24, and 32 h after CLP. All animals surviving CLP will be humanely sacrificed by isoflurane overdose and observed pneumothorax and necropsied immediately after the final bleeds at 32 h.

### Plasma Samples for LPS Measurements

Carotid arterial blood samples are obtained immediately before CLP (t=0 h), and at t=6 h, 24 h, and 32 h post CLP, or at the time of observed death (TOD) if this TOD is sooner than 32 h post-CLP. Carotid arterial blood samples are obtained in normal controls (without CLP) at the same time points. These arterial samples will be used for the LPS measurements in normal controls (no CLP) (n=25) and in animals with CLP (n=25). The approach to sample size estimation for the proposed project was based on the new test (HTRF LPS assay) having an area under the ROC curve (AUC) of 0.75, indicating good diagnostic properties. A sample size of 25 from the positive group and 25 from the negative group achieves 92% power when compared with an AUC of 0.5 (e.g., benchmark for chance alone) using a two-sided z-test at a significance level of 0.05. It is assumed standard deviations of measures from the positive and negative groups responses are equivalent. Power is higher (98%) if the AUC increases to 0.8.

Prior to sampling the carotid arterial catheter port is prepped with betadine. At each time point shown above 1.0 mL arterial blood is drawn and added to vials as per the manufacturer's instructions for the EAA assay to measure LPS in whole blood. Whole blood samples are tested with the EAA according to the manufacturer's instructions. A second arterial bleed of 0.4 mL at the same time point is collected in a 1 mL syringe containing K₃EDTA and placed immediately on ice or in a refrigerator at 4° C. for no greater than 1 h. This blood is centrifuged at 3000 rpm for 15 minutes at 4° C. to obtain at least 0.2 mL of plasma. The plasma is aseptically transferred with sterile pipettes to sterile cryovialsto measure LPS in plasma in the HTRF LPS assay. For the HTRF LPS assay LPS is extracted from plasma using the perchloric acid method (Obayashi, J Lab Clin Med 104:321-330, 1984). Following each arterial sampling 3× the volume of blood withdrawn is replaced by sterile NS via the jugular vein catheter.

### Diagnostic Sensitivity and Specificity for the HTRF LPS Assay Versus the EAA LPS Assay

LPS measurements are determined from standard curves for LPS generated for each assay, that is for the HTRF or EAA assay. True negative, true positive, false negative, and false positive measurements are determined for animals with and without CLP. Diagnostic sensitivity, and diagnostic specificity are calculated for the HTRF and the EAA assay and analyzed with the use of the Student's t test for unpaired data.

Statistical Significance: Data are Analyzed with the Student's t Test for Unpaired Data; Level of Significance is Set at P<0.05.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawing. Consequently, while the present invention has been described with reference to particular embodiments, modifications of structure, sequence, materials and the like apparent to those skilled in the art still fall within the scope of the invention as claimed by the applicant.

What is claimed is:

1. A method for measuring quantitative LPS levels in a sample using an A1 adenosine receptor TRF assay comprising:
a) selecting a source for an A1 adenosine receptor protein;
b) selecting at least one TRF fluorophore that is bound to a moiety selected from the group comprising:
i. a tag;
ii. a metabolic label;
iii. a protein label;
iv. an antibody to a tag, metabolic label, or protein label;
v. an antibody for the A1 adenosine receptor protein or peptide;
vi. an A1 adenosine receptor protein or peptide;
vii. a protein or peptide in an A1 adenosine receptor signaling pathway;
viii. an antibody to a protein or a peptide in an A1 adenosine receptor signaling pathway;
ix. an A1 adenosine receptor ligand;
x. a signaling molecule;
xi. a molecule in an A1 adenosine receptor signaling pathway;
xii. an antibody to a molecule in an A1 adenosine receptor signaling pathway; and
xiii. a molecule that can be measured in an A1 adenosine receptor signaling pathway; and
c) running the TRF assay utilizing the source for the A1 adenosine receptor protein; the at least one TRF fluorophore that is bound; and the sample in a manner that measurement of fluorescence correlates with the quantitative level of LPS.

2. A method according to claim 1 wherein the fluorophore is a lanthanide fluorophore.

3. A method according to claim 2 wherein the lanthanide fluorophore is europium.

4. A method according to claim 2 or 3 wherein the fluorophore is chelated to GTP.

5. A method according to claim 1 wherein the assay is a GTP binding assay.

6. A method according to claim 1 wherein the fluorophore is bound to a moiety selected from GTP, A1 adenosine receptor protein or peptide, an antibody for an A1 adenosine receptor protein or peptide, a tag, a protein label, an antibody to a tag or protein label, thromboxane, and cAMP.

7. A method according to claim 1 wherein the A1 adenosine receptor protein is expressed in a cell or a cell membrane.

8. A method according to claim 1 wherein the assay is a heterogeneous TRF assay.

9. A method according to claim 1 wherein the assay is a homogeneous TRF assay.

10. A method according to claim 1 wherein the sample is a biological sample.

11. A method according to claim 1 wherein the sample is a non-biological sample.

12. A method according to claim 1 which further comprises diagnosing the presence or activity of disease selected from the group comprising sepsis, Gram-negative bacterial infection, and LPS-related condition in a patient comprising measuring the amount of LPS in the sample from a standard curve for LPS generated by the TRF assay of claim 1.

13. A method for measuring quantitative LPS levels in a sample using an A1 adenosine receptor TRF assay comprising:
a) selecting source for an A1 adenosine receptor protein;
b) selecting a first and second TRF fluorophore that is each bound to a different moiety selected from the group comprising:
i. a tag;
ii. a metabolic label;
iii. a protein label;
iv. an antibody for a tag, metabolic label, or protein label;
v. an antibody for the A1 adenosine receptor protein or peptide;
vi. an A1 adenosine receptor protein or peptide;
vii. a protein or peptide in an A1 adenosine receptor signaling pathway;
viii. an antibody to a protein or peptide in an A1 adenosine receptor signaling pathway;
ix. an A1 adenosine receptor ligand;
x. a signaling molecule;
xi. a molecule in A1 adenosine receptor signaling pathway;
xii. an antibody to a molecule in an A1 adenosine receptor signaling pathway; and
xiii. a molecule that can be measured in an A1 adenosine receptor signaling pathway; selected such there is energy transfer between the two fluorophores following excitation in a TRF assay which can be measured; and
c) running the TRF assay utilizing the source for the A1 adenosine receptor protein; the first and second TRF fluorophores that are bound; and the sample in a manner that measurement of fluorescence correlates with the quantitative level of LPS.

14. A method according to claim 13 wherein the fluorophore is bound to a moiety selected from GTP, A1 adenosine receptor protein or peptide, an antibody for an A1 adenosine receptor protein or peptide, a tag, a protein label, an antibody to a tag or protein label, thromboxane, and cAMP.

15. A method according to claim 13 wherein the A1 adenosine receptor is expressed in a cell or a cell membrane.

16. A method according to claim 13 wherein the sample is a biological sample.

17. A method according to claim 13 wherein the sample is a non-biological sample.

18. A method according to claim 13 which further comprises diagnosing the presence or activity of disease selected from the group comprising sepsis, Gram-negative bacterial infection, and LPS-related condition in a patient comprising measuring the amount of LPS in the sample from a standard curve for LPS generated by the TRF assay of claim 13.

19. A method of diagnosing a patient for the presence of sepsis, a Gram-negative bacterial infection, or an LPS-related condition comprising measuring LPS in a biological sample from the patient comprising:
a) selecting a source for an A1 adenosine receptor protein;
b) selecting a quantitative TRF assay that utilizes at least one TRF fluorophore wherein the assay is quantitative for LPS;
c) running the assay with the sample;
d) measuring the amount of fluorescence for the sample in the assay;
e) determining the amount of LPS in the sample from the amount of fluorescence by comparing the fluorescence measurement for the sample to a standard curve for LPS generated by the assay of steps a) through d) with the use of samples spiked with known amounts of LPS; and
f) diagnosing the patient's condition from the amount of LPS in the biological sample from the patient.

20. A method according to claim 19 wherein the assay is a heterogeneous TRF assay.

21. A method according to claim 19 wherein the assay is a homogeneous TRF assay.

22. A method according to claim 19 wherein the assay is a GTP binding assay.

23. A method according to claim 19 wherein the fluorophore is bound to a moiety selected from GTP, A1 adenosine receptor protein or peptide, an antibody for an A1 adenosine receptor protein or peptide, a tag, a protein label, an antibody for a tag or protein label, thromboxane, and cAMP.

24. A method according to claim 19 wherein the A1 adenosine receptor is expressed in a cell or a cell membrane.

25. A kit for determination of LPS level in a sample comprising:
a) a selected TRF assay utilizing at least one fluorophore;
b) a source for the A1 adenosine receptor protein; and
c) an LPS standard.

26. A kit according to claim 25 wherein the fluorophore is a lanthanide fluorophore chelated to GTP or streptavidin designed for a GTP binding assay.

27. A kit according to claim 25 where the TRF assay is a heterogeneous assay.

28. A kit according to claim 25 wherein the TRF assay is a homogeneous assay.
